(19) United States
(12) Patent Application Publication
(10) Pub. No.: US 2017/0188891 A1
(43) Pub. Date: Jul. 6, 2017

(54) METHOD AND DEVICE FOR RETRIEVING A BREATHING SIGNAL

(71) Applicant: National Chung Cheng University, Min-Hsiung (TW)

(72) Inventors: Tay-Jyi LIN, Minhsiung Township (TW); Bo-Yuan YANG, Min-Hsiung (TW); Yi-Hsuan TING, Taichung City (TW); Ching-Wei YEH, Min-Hsiung (TW)

(21) Appl. No.: 15/083,759

(22) Filed: Mar. 29, 2016

(30) Foreign Application Priority Data
Dec. 31, 2015 (TW) 104144611

(51) Int. Cl. A61B 5/113

(52) U.S. Cl. CPC A61B 5/113

(57) ABSTRACT

A method and a device for retrieving a breathing signal is disclosed. Firstly, a triaxial acceleration signal is retrieved for a fixed period. Then, multivariate empirical mode decomposition (MEMD) is performed on the triaxial acceleration signal, so as to sequentially obtain a plurality of intrinsic mode functions (IMFs). Finally, an average inclination angle of the triaxial acceleration signal corresponding to each intrinsic mode function is sequentially calculated, and the intrinsic mode function corresponding to the average inclination angle within a predetermined angle range is added to a set. All the intrinsic mode functions are added up to obtain and output a breathing signal when an amount of the intrinsic mode functions in the set equals to a predetermined value being a natural number.

This application claims priority for Taiwan patent application no. 104144611 filed on Dec. 31, 2015, the content of which is incorporated in its entirely.

### BACKGROUND OF THE INVENTION

Field of the Invention

The present invention relates to a retrieving technology, particularly to a method and a device for retrieving a breathing signal.

Description of the Related Art

For round transportation of humans, vehicles are important traffic tools. With the popularity of vehicles, traffic accidents occur. Since modern people do not get enough sleep or have good sleep quality at night, they feel tired. In such a case, people only just drive so that “weary drive” easily occurs to result in traffic accidents. However, people value drink drive than weary drive without knowing seriousness of weary drive. The hurt caused by weary drive is as fatal as the hurt caused by drink drive. When a driver feels tired and asleep, his response to the outside is slowed, thereby affecting alertness and determination of driving condition. In many weary drive accidents, there is no brake track on the road. As a result, the weary drive accidents are very serious.

Recently, the study pointed out that synchronizing breathing with heartbeat can refresh a driver when the driver is weary with driving. Thus, when the driver drives, the breathing signal of the driver in a vehicle is measured. After immediately retrieving the breathing signal, the driving physiological information can be applied to the training of synchronization of breathing to heartbeat to refresh a driver lest weary drive occur. As a result, the measured breathing signal, which is the very important information for the physiological state of the driver in driving, refreshes a driver when the driver is weary with driving, thereby achieving driving safety.

Due to the importance of the breathing signal, there are many methods for measuring the breathing signal with the advancement of technology. Since the methods and principles thereof are different, their advantages and limitation are different. The common technologies for retrieving a breathing signal in the market and their instruments, advantages and limitations are introduced below.

A band-tying retrieving technology uses plastic textiles able to hug a participant. The plastic textiles are tied to the chest or the abdomen of the participant. The periodic heaving signal of the chest or the abdomen is retrieved according to the volume variation due to the fact that the chest or the abdomen heaves while breathing. Then, the resistance variation of the piezoelectric material installed on the plastic textiles or a weave is converted into an electrical signal as a breathing signal whose waveform is shown in FIG. 1. The vertical axis of FIG. 1 has an arbitrary unit (AU). Since the band-tying retrieving technology can directly detect the heave of the chest and the material used is conveniently obtained, the technology is a wide measuring method in the market.

This technology uses contact, compression and expansion to provide pressure to the resistance of the piezoelectric material or weave of tying bands. In order to accurately measure the breathing signal, a user has to tighten the chest or the abdomen with the plastic textiles of the tying bands whereby the tying bands hug the participant. If the plastic textiles loose, there is no pressure caused by breathing of the participant on the resistance of the piezoelectric material or weave of tying bands. Thereby, the breathing signal of the participant cannot be measured. However, tightening the chest or the abdomen of the participant makes the user feel uncomfortable when the user is monitored for a long time. As a result, the technology is not suitable for long-term breathing monitoring.

A radar retrieving technology mainly uses radars to emit electromagnetic waves transmitted to the chest. The variations of the wavelengths and phases of the reflection waves are caused by the heave of the chest in breathing. The difference of the reflection waves and initial waves is compared to detect the micro-heave of the chest, which is called coherent demodulation. Based on the Doppler effect, the breathing signal is figured out. Since the principle of using the radars to emit electromagnetic waves is used, the measuring method can achieve precise detection. The radar retrieving technology is very promising.

However, the technology requires expensive equipment and complicated devices. The equipment is not easy to be carried on people or installed on vehicles. With the advancement of technology, a volume of a radar-emitting device is smaller and smaller. But the cost of the device is still higher. Most of the users concerned the affection on the health of human bodies when the human bodies are irradiated by radars for a long time.

An image-captured retrieving technology uses a webcam or a camera lens to capture the brightness variation produced by blood vessels of the face that blood flows, so as to figure out the pulse and the breathing signal of a human body. Alternatively, the technology directly captures the chest or the abdomen of a human body, and then uses depth estimation and stereo imaging techniques to determine the heave of the chest or the abdomen of the human body when breathing, thereby figuring out the breathing signal. The cost of the technology is very low since the technology can uses a cheap camera to retrieve the breathing signal. In addition, most of the mobile phones have camera lenses. As a result, the camera is easily obtained. It is very convenient to measure the breathing signal.

Although the technology has the low cost, the technology is related to capturing portraits. As a result, a private issue of users is worth discussion. The technology is strict with an illumination environment. In a dark environment, the technology may determine incorrectly or not be used. If the camera is installed on a vehicle, the measuring method is ineffective at night or in a cave, which limits applications of measuring the breathing signal.

For an electrocardiography (ECG) determination retrieving technology, a gas exchange process between a human body and the outside is performed by using blood to carry oxygen to cells of the human body when the human body breathes. The heart squeezes so that blood can circulate in the body. From this concept, it is known that the specific pulsing relation exists between heartbeat and breathing Hence, a breathing signal is determined by analyzing an ECG signal.

Since the breathing signal is determined by analyzing the ECG signal, the depth and strength of breathing cannot be directly determined, which limits the related applications, such as analysis and training of abdominal breathing or application for refreshing of weary drive based on synchronization of an ECG signal to a breathing signal.

A US patent NO. 20120296221, a U.S. Pat. No. 5,309,922, a US patent NO. 20110066041 and a US patent NO. 20100030085 all use accelerometers to measure a breathing signal for the heave of the chest. Since the accelerometer has high sensitivity and is easily affected by acceleration excluding breathing, the largest limitation of using the accelerometer to measure the breathing signal is to require an additional signal-processing method for dealing with acceleration of noise. The four US patents use filters to filter out signals. However, before the filter filters out the signals, the characteristics of the signals are clearly understood. The US patent NO. 20120296221 detects and analyzes noise and then filters it out. In the U.S. Pat. No. 5,309,922, the US patent NO. 20110066041 and the US patent NO. 20100030085, the characteristic of noise is predetermined and then the noise is filtered out. However, understand the characteristic of signals to retrieve the breathing signal is troublesome.

To overcome the abovementioned problems, the present invention provides a method and a device for retrieving a breathing signal, so as to solve the afore-mentioned problems of the prior art.

### SUMMARY OF THE INVENTION

A primary objective of the present invention is to provide a method and a device for retrieving a breathing signal, which uses a triaxial acceleration sensor to retrieve a triaxial acceleration signal, performs multivariate empirical mode decomposition (MEMD) on the triaxial acceleration signal, and automatically obtains a breathing signal without understanding the spectrum characteristics of the signal in advance and human intervention. Besides, the triaxial acceleration sensor has the advantages of low cost, comfortable wear, convenient installment and without private issues.

To achieve the abovementioned objectives, the present invention provides a method for retrieving a breathing signal. Firstly, a triaxial acceleration signal is retrieved by a triaxial acceleration sensor for a fixed period. For example, the triaxial acceleration sensor is a triaxial accelerometer arranged on a safe belt. Then, multivariate empirical mode decomposition (MEMD) is performed on the triaxial acceleration signal, so as to sequentially obtain a plurality of intrinsic mode functions (IMFs). Finally, an average inclination angle of the triaxial acceleration signal corresponding to each intrinsic mode function is sequentially calculated, and the intrinsic mode function corresponding to the average inclination angle within a predetermined angle range is added to a set. All the intrinsic mode functions are added up to obtain and output a breathing signal when an amount of the intrinsic mode functions in the set equals to a predetermined value being a natural number.

The average inclination angle is an average value of a plurality of inclination angles θₜ, and each inclination angle θₜ is obtained by triaxial acceleration vectors of the corresponding triaxial acceleration signal at time points t and (t−1) during the fixed period, and the triaxial acceleration vector at time point t is (xₜ, yₜ, zₜ), and the triaxial acceleration vector at time point (t−1) is (xₜ₋₁, yₜ₋₁, zₜ₋₁), and each inclination angle

$${θ}_{t} = {cos}^{- 1} ( \frac{{x}_{t} {x}_{t - 1} + {y}_{t} {y}_{t - 1} + {z}_{t} {z}_{t - 1}}{\sqrt{{x}_{t}^{2} + {y}_{t}^{2} + {z}_{t}^{2}} \sqrt{{x}_{t - 1}^{2} + {y}_{t - 1}^{2} + {z}_{t - 1}^{2}}} ) .$$

The present invention also provides a device for retrieving a breathing signal. The device includes a triaxial acceleration sensor and a processor. For example, the triaxial acceleration sensor is a triaxial accelerometer arranged on a safe belt. Besides, the triaxial acceleration sensor and the processor are integrated in a smart phone. The triaxial acceleration sensor retrieves a triaxial acceleration signal for a fixed period and outputs the triaxial acceleration signal. The processor is connected with said triaxial acceleration sensor, receives the triaxial acceleration signal, performs multivariate empirical mode decomposition (MEMD) on the triaxial acceleration signal, so as to sequentially obtain a plurality of intrinsic mode functions (IMFs), sequentially calculates an average inclination angle of the triaxial acceleration signal corresponding to each intrinsic mode function, adds the intrinsic mode function corresponding to the average inclination angle within a predetermined angle range to a set, and adds up all the intrinsic mode functions to obtain and output a breathing signal when an amount of the intrinsic mode functions in the set equals to a predetermined value being a natural number.

The average inclination angle is an average value of a plurality of inclination angles θₜ, and each inclination angle θₜ is obtained by triaxial acceleration vectors of the corresponding triaxial acceleration signal at time points t and (t−1) during the fixed period, and the triaxial acceleration vector at time point t is (xₜ, yₜ, zₜ), and the triaxial acceleration vector at time point (t−1) is (xₜ₋₁, yₜ₋₁, zₜ₋₁), and each inclination angle

$${θ}_{t} = {cos}^{- 1} ( \frac{{x}_{t} {x}_{t - 1} + {y}_{t} {y}_{t - 1} + {z}_{t} {z}_{t - 1}}{\sqrt{{x}_{t}^{2} + {y}_{t}^{2} + {z}_{t}^{2}} \sqrt{{x}_{t - 1}^{2} + {y}_{t - 1}^{2} + {z}_{t - 1}^{2}}} ) .$$

Below, the embodiments are described in detail in cooperation with the drawings to make easily understood the technical contents, characteristics and accomplishments of the present invention.

### BRIEF DESCRIPTION OF THE DRAWINGS

FIG. 1 is a diagram schematically showing a waveform of a breathing signal in the conventional technology;

FIG. 2 is a block diagram schematically showing a device for retrieving a breathing signal according to an embodiment of the present invention;

FIG. 3 is a flow chart showing a method for retrieving the breathing signal according to an embodiment of the present invention;

FIG. 4 is a diagram schematically showing waveforms of a triaxial acceleration signal in X, Y and Z axes according to an embodiment of the present invention;

FIG. 5 is a diagram schematically showing waveforms of the 18^{th} intrinsic mode function in X, Y and Z axes according to an embodiment of the present invention;

FIG. 6 is a diagram schematically showing waveforms of the 19^{th} intrinsic mode function in X, Y and Z axes according to an embodiment of the present invention;

FIG. 7 is a diagram schematically showing waveforms of the 20^{th} intrinsic mode function in X, Y and Z axes according to an embodiment of the present invention;

FIG. 8 is a diagram schematically showing waveforms of the 21^{th} intrinsic mode function in X, Y and Z axes according to an embodiment of the present invention;

FIG. 9 is a diagram schematically showing waveforms of the 22^{th} intrinsic mode function in X, Y and Z axes according to an embodiment of the present invention;

FIG. 10 is a diagram schematically showing waveforms of another triaxial acceleration signal in X, Y and Z axes according to an embodiment of the present invention; and

FIG. 11 is a diagram schematically showing a waveform of a breathing signal according to an embodiment of the present invention.

### DETAILED DESCRIPTION OF THE INVENTION

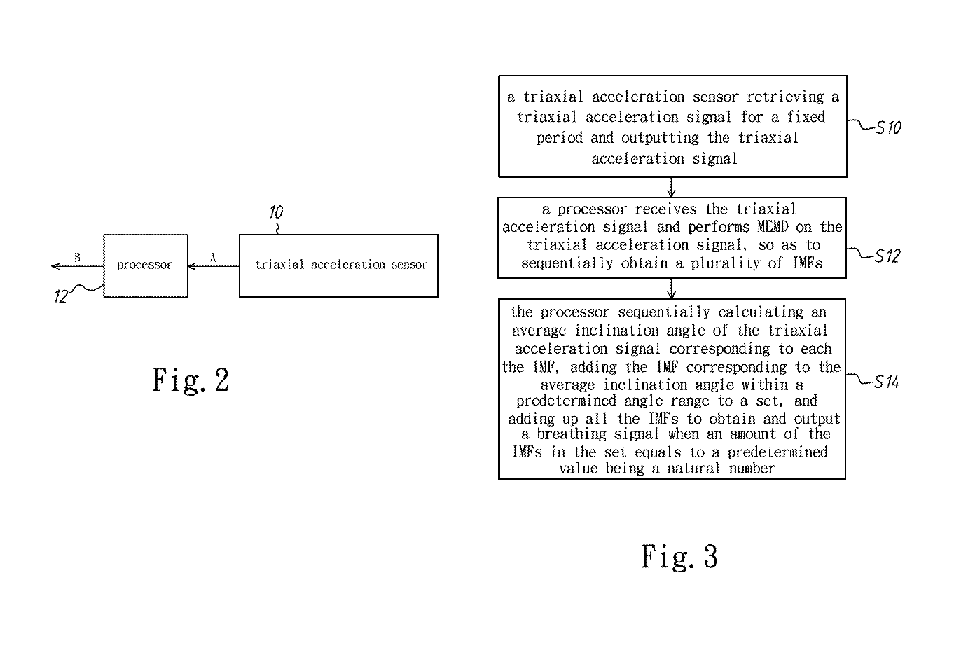

Refer to FIG. 2. A device for retrieving a breathing signal of the present invention comprises a triaxial acceleration sensor 10 and a processor 12, wherein the triaxial acceleration sensor 10 is realized with a triaxial accelerometer having low cost instead of uncomfortable tying bands. Since the triaxial accelerometer is usually built in a smart phone easily obtained, the triaxial acceleration sensor 10 and the processor 12 are also integrated in the smart phone whereby the acceleration sensor 10 operates in cooperation with the processor 12. In addition to the low cost, the triaxial acceleration sensor 10 has the advantages of comfortable wear, convenient installment and without private issues. When a vehicle drives, fastening a seat belt is an essential safety precaution. In Taiwan, fastening a seat belt is prescribed in the law. As a result, when a vehicle drives, a driver has to fasten a seat belt. The seat belt is pressed against the chest of the driver. Thus, the triaxial acceleration sensor 10 is arranged on the seat belt to sense the acceleration of the heave of the chest of the driver when the driver breathes. On the other hand, when the triaxial acceleration sensor 10 and the processor 12 are also integrated in the smart phone, the smart phone is arranged on a seat belt to measure a breathing signal of the driver whereby the breathing signal is applied to health care and monitoring.

The triaxial acceleration sensor 10 retrieves a triaxial acceleration signal A for a fixed period and outputs the triaxial acceleration signal A. The processor 12 is connected with the triaxial acceleration sensor 10, receives the triaxial acceleration signal A, and performs multivariate empirical mode decomposition (MEMD) on the triaxial acceleration signal, so as to sequentially obtain a plurality of intrinsic mode functions (IMFs), namely IMF(1), IMF(2) . . . IMF(n), wherein n is an index of the IMF. IMF(n) is the nth IMF. The processor 12 sequentially calculates an average inclination angle of the triaxial acceleration signal corresponding to each intrinsic mode function, and adds the intrinsic mode function corresponding to the average inclination angle within a predetermined angle range to a set, and adds up all the intrinsic mode functions to obtain and output a breathing signal B when an amount of the intrinsic mode functions in the set equals to a predetermined value being a natural number. The predetermined value is the amount that the IMF consisting of the breathing signal B requires. In other words, the present invention automatically and effectively obtains the breathing signal in a driving environment with high acceleration without understanding the spectrum characteristics of the signal in advance and human intervention. In addition, since the present invention does not use a heartbeat signal to determine the breathing signal, the present invention helps driver prevent from weary drive in driving without limiting biomedical applications based on synchronization of an electrocardiography (ECG) signal to a breathing signal.

Each IMF has N data points during the fixed period. Thus, each IMF has (N−1) inclination angles, wherein N is a natural number larger than or equal to 2. As a result, each IMF has an average inclination angle in physical movement. The average inclination angle is an average value of a plurality of inclination angles θₜ, and each inclination angle θₜ is obtained by triaxial acceleration vectors of the corresponding triaxial acceleration signal at time points t and (t−1) during the fixed period, and the triaxial acceleration vector at time point t is (xₜ, yₜ, zₜ), and the triaxial acceleration vector at time point (t−1) is (xₜ₋₁, yₜ₋₁, zₜ₋₁), and each inclination angle θₜ is expressed by a formula (1):

$${θ}_{t} = {cos}^{- 1} ( \frac{{x}_{t} {x}_{t - 1} + {y}_{t} {y}_{t - 1} + {z}_{t} {z}_{t - 1}}{\sqrt{{x}_{t}^{2} + {y}_{t}^{2} + {z}_{t}^{2}} \sqrt{{x}_{t - 1}^{2} + {y}_{t - 1}^{2} + {z}_{t - 1}^{2}}} ) \qquad ( 1 )$$

Refer to FIG. 2 and FIG. 3. The operation of the present invention is introduced below. Firstly, in Step S10, the triaxial acceleration sensor 10 retrieves the triaxial acceleration signal A for a fixed period and outputs the triaxial acceleration signal A. Then, in Step S12, the processor 12 receives the triaxial acceleration signal A, and performs multivariate empirical mode decomposition (MEMD) on the triaxial acceleration signal, so as to sequentially obtain a plurality of intrinsic mode functions IMF(1), IMF(2) . . . IMF(n) having different meaning Since the IMFs have different meaning, the IMFs have different inclination angles θₜ. Besides, when a human body breathes, the inclination angle that the chest heaves is within a range. Thus, the present invention uses the property to recognize the breathing signal B and view the signals having inclination angles without the range as noise and excludes the noise. After Step S12, Step S14 is performed. In Step S14, the processor 12 sequentially calculates an average inclination angle of the triaxial acceleration signal corresponding to each intrinsic mode function, and adds the intrinsic mode function corresponding to the average inclination angle within the predetermined angle range to a set, and adds up all the intrinsic mode functions to obtain and output the breathing signal B when an amount of the intrinsic mode functions in the set equals to the predetermined value.

For example, suppose the predetermined value equals to three. In Step S14, the processor 12 firstly calculates the average inclination angle of the first intrinsic mode function IMF(1), observes that the average inclination angle of IMF(1) is within the predetermined angle range and adds IMF(1) to the set. Then, the processor 12 calculates the average inclination angle of the second intrinsic mode function IMF(2), observes that the average inclination angle of IMF(2) is within the predetermined angle range and adds IMF(2) to the set. Then, the processor 12 calculates the average inclination angle of the third intrinsic mode function IMF(3), observes that the average inclination angle of IMF(3) is without the predetermined angle range and excludes IMF(3) from the set. Finally, the processor 12 calculates the average inclination angle of the second intrinsic mode function IMF(4), observes that the average inclination angle of IMF(4) is within the predetermined angle range and adds IMF(4) to the set. At this time, Since the amount of the IMFs in the set has to equaled to the predetermined value, the processor 12 adds up IMF(1), IMF(2) and IMF(4) to obtain and output the breathing signal.

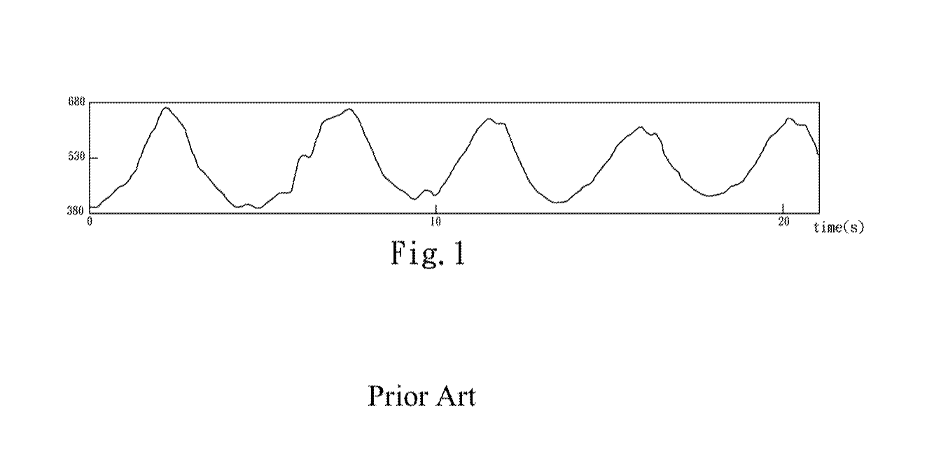

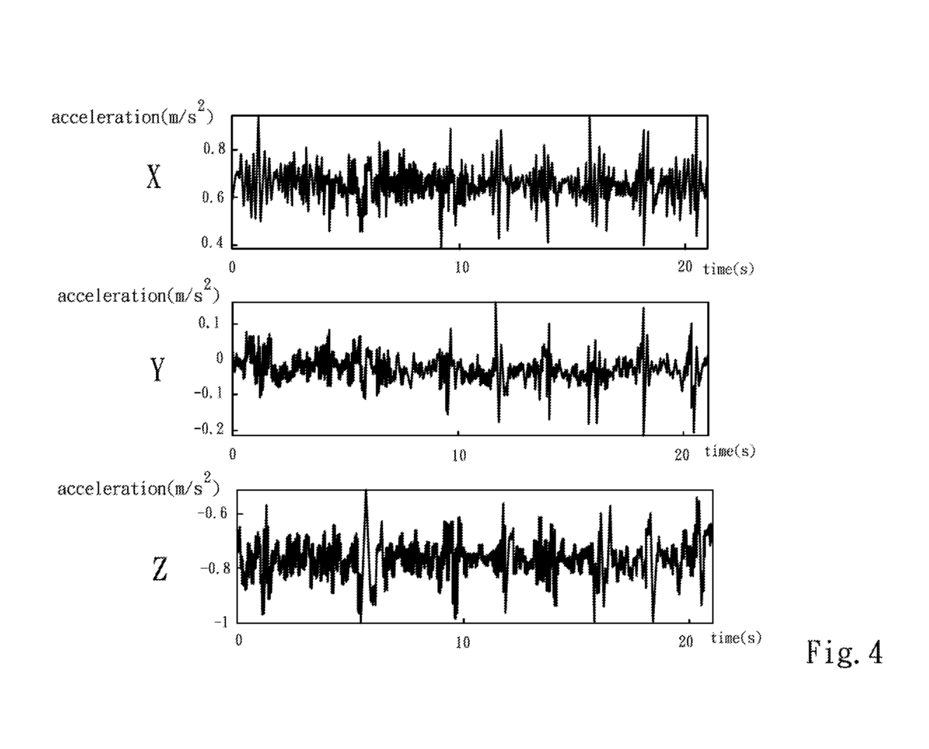

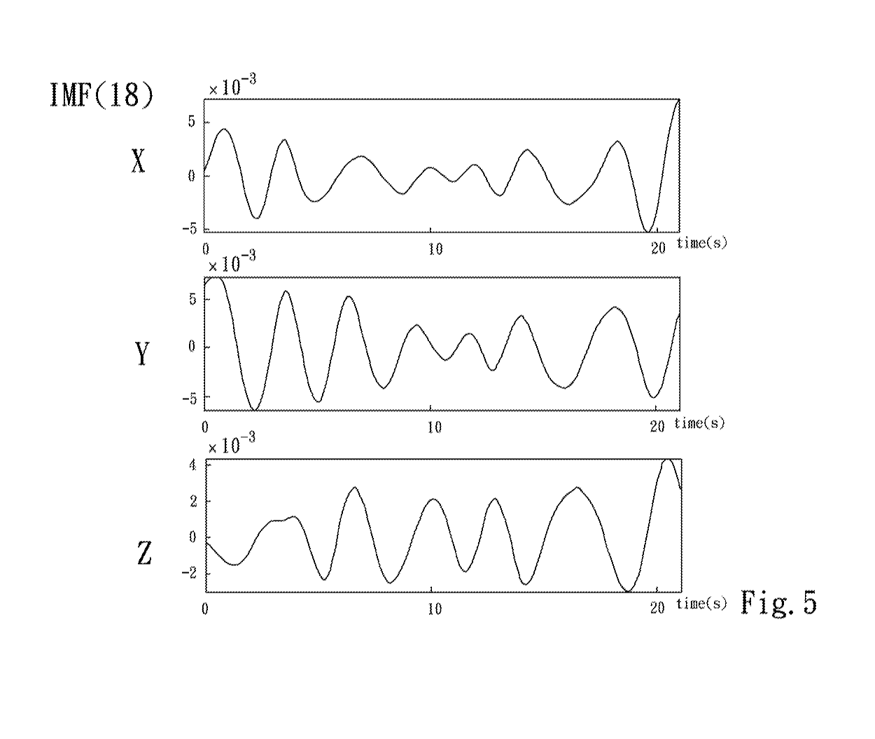

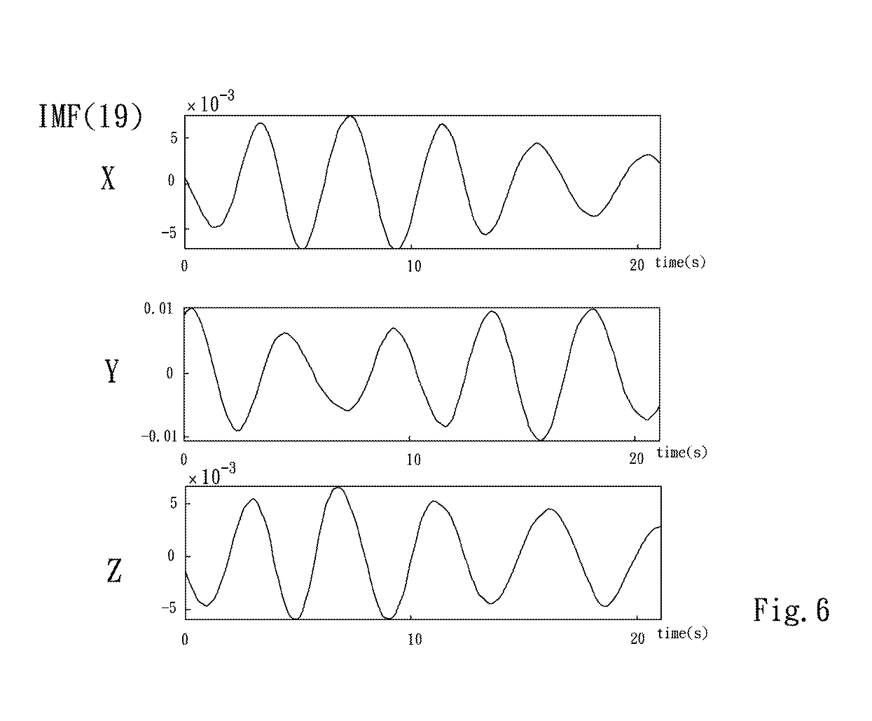

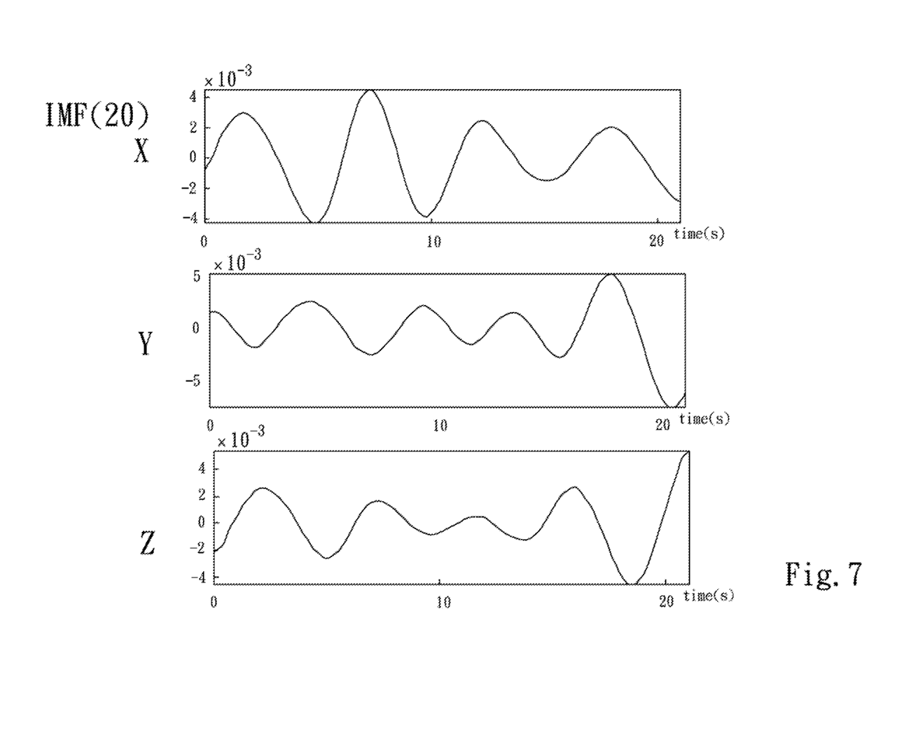

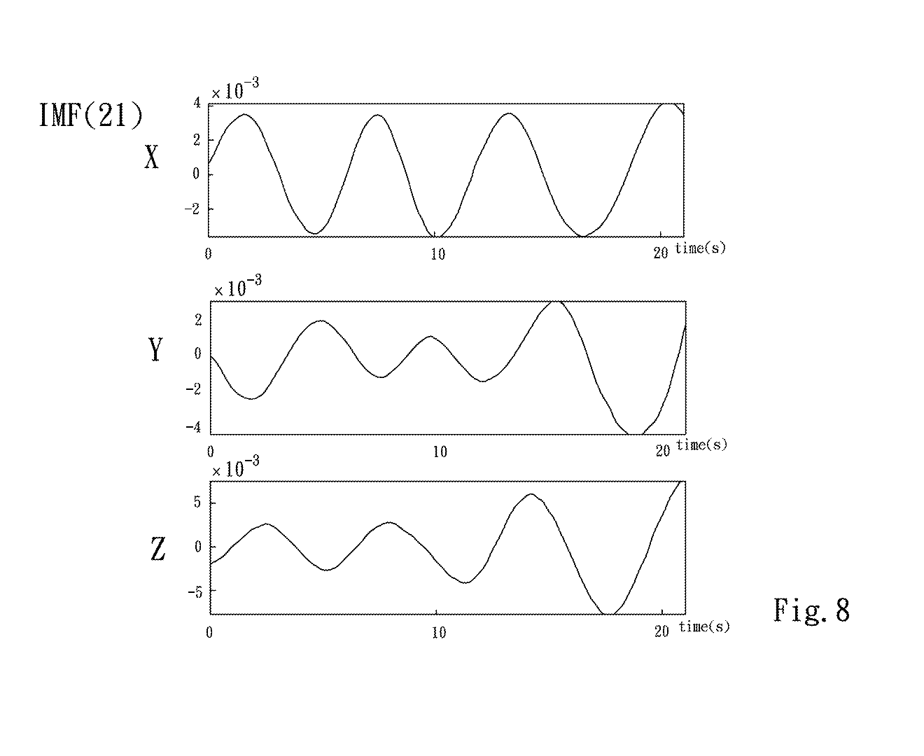

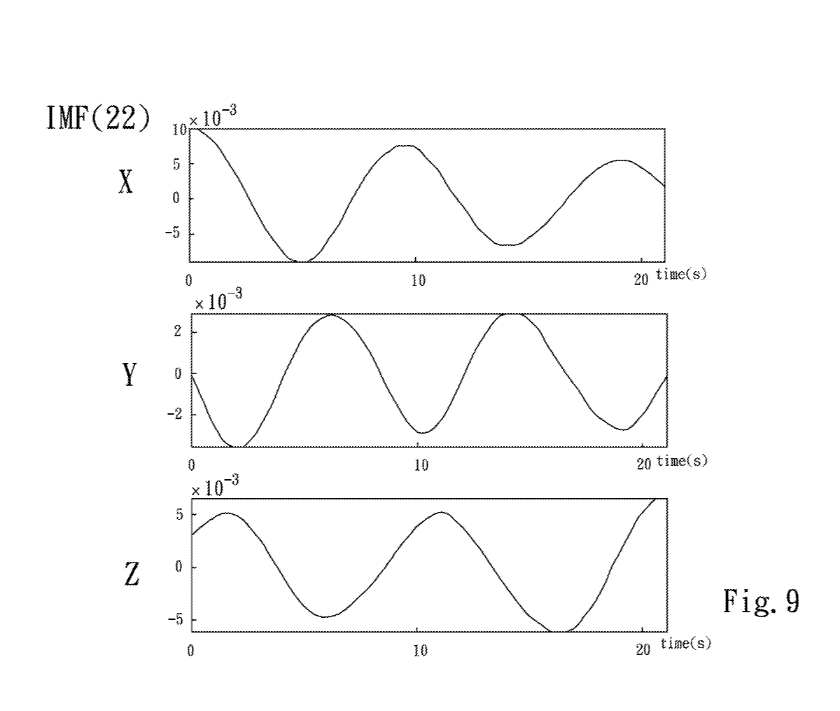

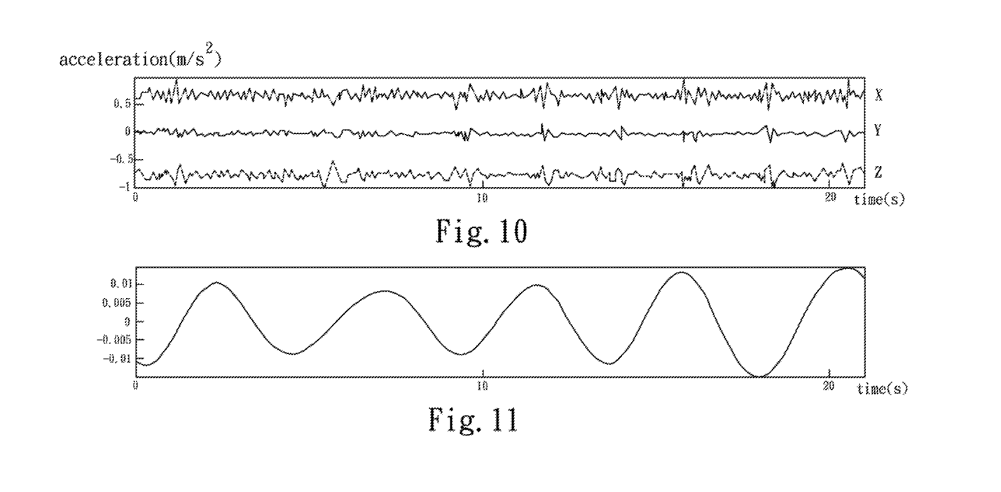

FIG. 4 is a diagram schematically showing waveforms of a triaxial acceleration signal in X, Y and Z axes measured by a triaxial accelerometer installed on a seat belt according to an embodiment of the present invention, wherein the triaxial acceleration signal includes an acceleration signal in X, Y and Z axes. Besides, the 18^{th}, 19^{th}, 20^{th}, 21^{th} and 22^{th} intrinsic mode functions IMF(18), IMF(19), IMF(20), IMF(21) and IMF(22) corresponding to the triaxial acceleration signal are respectively shown in FIG. 5, FIG. 6, FIG. 7, FIG. 8 and FIG. 9. Each IMF includes signals in X, Y and Z axes. FIG. 10 is a diagram schematically showing waveforms of another triaxial acceleration signal in X, Y and Z axes measured by the triaxial accelerometer installed on the seat belt according to an embodiment of the present invention, wherein the triaxial acceleration signal includes an acceleration signal in X, Y and Z axes. FIG. 11 is a diagram schematically showing a waveform of a breathing signal according to an embodiment of the present invention. Compared with FIG. 1, the breathing signal of FIG. 11 is more precise.

In conclusion, the present invention uses the triaxial acceleration sensor having the advantages of low cost, comfortable wear, convenient installment and without private issues, and uses MEMD to automatically obtain the breathing signal without understanding the spectrum characteristics of the signal in advance and human intervention.

The embodiments described above are only to exemplify the present invention but not to limit the scope of the present invention. Therefore, any equivalent modification or variation according to the shapes, structures, features, or spirit disclosed by the present invention is to be also included within the scope of the present invention.

What is claimed is:

1. A method for retrieving a breathing signal comprising steps of:
retrieving a triaxial acceleration signal for a fixed period;
performing multivariate empirical mode decomposition (MEMD) on said triaxial acceleration signal, so as to sequentially obtain a plurality of intrinsic mode functions (IMFs); and
sequentially calculating an average inclination angle of said triaxial acceleration signal corresponding to each said intrinsic mode function, and adding said intrinsic mode function corresponding to said average inclination angle within a predetermined angle range to a set, and adding up all said intrinsic mode functions to obtain and output a breathing signal when an amount of said intrinsic mode functions in said set equals to a predetermined value being a natural number.

2. The method for retrieving the breathing signal of claim 1, wherein said average inclination angle is an average value of a plurality of inclination angles θₜ, and each said inclination angle θₜ is obtained by triaxial acceleration vectors of corresponding said triaxial acceleration signal at time points t and (t−1) during said fixed period, and said triaxial acceleration vector at time point t is (xₜ, yₜ, zₜ), and said triaxial acceleration vector at time point (t−1) is (xₜ₋₁, yₜ₋₁, zₜ₋₁), and each said inclination angle
$${θ}_{t} = {cos}^{- 1} ( \frac{{x}_{t} {x}_{t - 1} + {y}_{t} {y}_{t - 1} + {z}_{t} {z}_{t - 1}}{\sqrt{{x}_{t}^{2} + {y}_{t}^{2} + {z}_{t}^{2}} \sqrt{{x}_{t - 1}^{2} + {y}_{t - 1}^{2} + {z}_{t - 1}^{2}}} ) .$$

3. The method for retrieving the breathing signal of claim 1, wherein said triaxial acceleration signal is retrieved by a triaxial acceleration sensor.

4. The method for retrieving the breathing signal of claim 3, wherein said triaxial acceleration sensor is a triaxial accelerometer.

5. The method for retrieving the breathing signal of claim 3, wherein said triaxial acceleration sensor is arranged on a safe belt.

6. A device for retrieving a breathing signal comprising:
a triaxial acceleration sensor retrieving a triaxial acceleration signal for a fixed period and outputting said triaxial acceleration signal;
a processor connected with said triaxial acceleration sensor, receiving said triaxial acceleration signal, performing multivariate empirical mode decomposition (MEMD) on said triaxial acceleration signal, so as to sequentially obtain a plurality of intrinsic mode functions (IMFs), sequentially calculating an average inclination angle of said triaxial acceleration signal corresponding to each said intrinsic mode function, adding said intrinsic mode function corresponding to said average inclination angle within a predetermined angle range to a set, and adding up all said intrinsic mode functions to obtain and output a breathing signal when an amount of said intrinsic mode functions in said set equals to a predetermined value being a natural number.

7. The device for retrieving the breathing signal of claim 6, wherein said average inclination angle is an average value of a plurality of inclination angles θₜ, and each said inclination angle θₜ is obtained by triaxial acceleration vectors of corresponding said triaxial acceleration signal at time points t and (t−1) during said fixed period, and said triaxial acceleration vector at time point t is (xₜ, yₜ, zₜ), and said triaxial acceleration vector at time point (t−1) is (xₜ₋₁, yₜ₋₁, zₜ₋₁), and each said inclination angle
$${θ}_{t} = {cos}^{- 1} ( \frac{{x}_{t} {x}_{t - 1} + {y}_{t} {y}_{t - 1} + {z}_{t} {z}_{t - 1}}{\sqrt{{x}_{t}^{2} + {y}_{t}^{2} + {z}_{t}^{2}} \sqrt{{x}_{t - 1}^{2} + {y}_{t - 1}^{2} + {z}_{t - 1}^{2}}} ) .$$

8. The device for retrieving the breathing signal of claim 6, wherein said triaxial acceleration sensor is a triaxial accelerometer.

9. The device for retrieving the breathing signal of claim 6, wherein said triaxial acceleration sensor is arranged on a safe belt.

10. The device for retrieving the breathing signal of claim 9, wherein said triaxial acceleration sensor and said processor are integrated in a smart phone.
